(19) United States
(12) Patent Application Publication
(10) Pub. No.: US 2022/0259279 A1
(43) Pub. Date: Aug. 18, 2022

(54) ALGINATE-BASED MICROCAPSULATION FOR THE DELIVERY OF ALPHA-CGRP IN CARDIOVASCULAR DISEASES

(71) Applicant: University of South Carolina, Columbia, SC (US)

(72) Inventors: Ambrish Kumar, Columbia, SC (US); Jay D. Potts, Columbia, SC (US); Donald J. Dipette, Blythewood, SC (US); Marwa Belhaj, Columbia, SC (US)

(73) Assignee: University of South Carolina, Columbia, SC (US)

(21) Appl. No.: 17/630,628

(22) PCT Filed: Jul. 31, 2020
(86) PCT No.: PCT/US2020/044407

§ 371 Date: Jan. 27, 2022

Related U.S. Application Data
(60) Provisional application No. 62/880,723, filed on Jul. 31, 2019.

(51) Int. Cl. C07K 14/585; A61K 9/50; A61P 9/08

(52) U.S. Cl. CPC C07K 14/585; A61P 9/08; A61K 9/5036

(57) ABSTRACT

Methods and systems for delivering a very potent vasodilator that has the ability to treat and prevent heart failure including delivering microcapsules containing α-CGRP, which show no toxicity and lowers blood pressure similar to the native peptide, where this new compound could greatly enhance the lifespan of patients suffering from heart failure.

### BACKGROUND OF THE INVENTION

1) Field of the Invention

The present invention relates to methods and systems for delivering a very potent vasodilator that has the ability to treat and prevent heart failure including delivering microcapsules containing α-CGRP, which show no toxicity and lowers blood pressure similar to the native peptide, where this new compound could greatly enhance the lifespan of patients suffering from heart failure.

2) Description of Related Art

The term cardiovascular disease (CVD) is used to describe a range of pathological conditions that affect the health of the heart and blood vessels. Some of the examples of CVD include: coronary artery disease, heart attack, heart failure, high blood pressure, hypertension, myocardial ischemia, myocardial infarction, and stroke. CVD is number one worldwide killer of men and women, including the United States. See, Benjamin et al., American Heart Association Council on E, Prevention Statistics C, Stroke Statistics S (2018) Heart Disease and Stroke Statistics-2018 Update: A Report From the American Heart Association. Circulation 137: e67-e492, 2018. It is estimated that nearly 1 in 3 deaths in the United States is attributed to CVD. In 2015, ˜41.5% of the U.S. population had at least one CVD condition, and in similar year the number of individuals affected by high blood pressure, coronary heart disease, stroke, congestive heart failure and atrial fibrillation was (in million) 96.1, 16.8, 7.5, 5.8, and 5.2, respectively (www.cdc.gov).

Since several years an important cardiovascular role for a peptide, alpha-calcitonin gene related peptide (α-CGRP), has been established in the inventors' laboratory, as well as others, in normal cardiovascular function and in a variety of cardiovascular diseases, including experimental hypertension, myocardial infarction, ischemic-reperfusion cardiac injury, and heart failure (Chai et al, 2006; Gangula et al, 1997; Huang et al, 2008; Katki et al, 2001; Li et al, 2013a; Li et al. 2013b; Supowit et al, 2005). α-CGRP is a 37-amino acid neuropeptide and is generated from the alternative splicing of the primary transcript of the calcitonin/α-CGRP gene CALC I (Breimer et al. 1988; Rosenfeld et al. 1983). α-CGRP synthesis is limited to specific regions of the central and peripheral nervous systems particularly in the sensory neurons of the dorsal root ganglia (DRG) which terminate peripherally on blood vessels (Russell et al, 2014). α-CGRP has markedly greater activity in the regulation of cardiovascular function (Brain et al, 1985). At cellular level, α-CGRP signals are mediated through its receptor known as the calcitonin receptor-like receptor (CLR). To be functionally active. CLR requires two accessory proteins—(i) Receptor Activity Modifying Protein (RAMP), and (ii) Receptor component Protein (RCP).

The RAMP family of proteins (RAMP-1, RAMP-2, and RAMP-3) are single domain transmembrane proteins and help in transporting CLR from the endoplasmic-reticulum/Golgi complex to the plasma membrane (McLatchie et al, 1998). α-CGRP has very specific binding affinity to CLR/RAMP-1 complex, while other neuropeptides, such as adrenomedullin, signal through CLR/RAMP-2 and CLR/RAMP-3 (Muff et al. 1.995). On other hand, RCP is a small intracellular peripheral membrane protein and remain associated with the loop region of CLR Peptide α-CGRP is the most potent vasodilator discovered to date and has positive chronotropic and inotropic effects (Brain et al, 1985; Supowit al. 1995). Systemic administration of α-CGRP, even at picomole concentration, lowers blood pressure in normotensive and hypertensive animals and humans (DiPette et. al. 1987; DiPette et al, 1989; Dubois-Rande et al, 1992; Supowit et al. 1993). Various in vivo and in vitro studies confirm that α-CGRP benefits the heart by decreasing angiotensin II activity, increasing cardiac blood flow through its potent vasodilator activity, and protecting cardiomyocytes from ischemia and metabolic stress (Rusell et al, 2014) ENREF 17. The inventors' laboratory has also demonstrated that α-CGRP acts as a compensatory depressor to attenuate the rise in blood pressure in three different models of experimental hypertension: 1) deoxycorticosterone (DOC)-salt (Supowit et al, 997), 2) subtotal nephrectomy-salt (Supowit et al, 1998), and 3) L-NAME induced hypertension during pregnancy (Gangula et al. 1997). A similar compensatory depressor role of α-CGRP has also been shown in the two-kidney one-clip model of hypertension (Supowit et al, 1997), and in chronic hypoxic pulmonary hypertension (Bivalacqua et al, 2002; Tjen et al, 1992).

A study from the inventors' laboratory showed that pressure-overload heart failure, induced by transverse aortic constriction (TAC), significantly exacerbates cardiac hypertrophy and subsequent cardiac dilation and dysfunction, cardiac fibrosis, and mortality in α-CGRP knock-out (KO) mice compared to their counterpart TAC wild-type mice (Li et al, 2013b). TAC α-CGRP KO mice hearts exhibit a dramatic increase in apoptosis, fibrosis, and inflammation in comparison to TAC wild-type mice, indicating that α-CGRP is critical to cardio-protection from pressure-overload induced congestive heart failure. Recently, the inventors studied the protective effect of exogenously administered α-CGRP in TAC heart failure mouse model. The inventors' in vivo studies confirm that α-CGRP delivery for 28 days, through mini-osmotic pump, protects the failing heart from TAC-induced pressure overload. In TAC-mice, α-CGRP administration significantly preserves the hearts at functional and anatomical levels by reducing cardiac cell death, fibrosis, and oxidative stress (Kumar et al, 2019. These studies indicated that α-CGRP is a promising drug candidate to treat cardiovascular diseases. However, peptide α-CGRP has very short half-life (t_{1/2}=˜5.5 min) in human plasma (Russell et al, 2014) as endopeptidases endothelin-converting enzyme-1 (ECE-1) and insulin-degrading enzyme (IDE) cleaves α-CGRP in the circulation (Hartopro et al, 2013; Kim et al. 2012). Hence, short half-life of peptide and non-applicability of mini-osmotic pumps in humans limit this approach to use α-CGRP as a drug for long-term treatment regime in humans.

In recent years, the pharmaceutical industry has been extensively using the U.S. Food and Drug Administration (US-FDA) approved alginate polymers as a novel drug carrier, and several clinical trials on alginate-based formulations are currently proceeding. Alginate is a water soluble linear polysaccharide and is isolated from the brown algae. Structurally it is unbranched polyanionic polysaccharides of 1-4 linked α-L-guluronic acid (G) and 8-D-mannuronic acid (M). As alginate polymer in stable at wide range of temperature (0-100° C.), non-toxic, and biocompatible, a wide range of molecules—from peptide, DNA, antibodies, protein to cells—have been used for encapsulation (Annamalai et al. 2018; Gu et al, 2004; Moore et at 2013a; Moore et al, 2014; Zhan et a, 2011). The inventors' laboratory has routinely utilized alginate-based drug delivery technology to encapsulate various proteins, inhibitors, and cells (Moore et al 2013a; Moore et al 2013b), and also reported that alginate microcapsules provide controlled release of a connexin-43 peptide, α-carboxy terminus-1, and rapidly closed the corneal wound closure in diabetic rats (Moore et al, 2014).

The American Heart Association (AHA) estimates that by 2035, 45.1% of the US population would have some form of CVD. The direct and indirect treatment cost of CVD in the USA continues to rise. In 2016, it was $555 billion and is expected to rise $1.1 trillion by 2035. Hence, placing a heavy financial burden on the economy and the health care system. Although there are several classes of drugs available to treat and prevent cardiac diseases, the 5-year survival rate is still only 50%. Thus, more effective therapeutic strategies are needed to be established. Further, non-applicability of osmotic pumps in humans and the short half-life of α-CGRP (˜5.5 min in the human plasma) limit this approach to use α-CGRP as a drug in humans. Accordingly, it is an object of the present invention to overcome this problem, and provide a novel drug delivery system for α-CGRP in order to maintain a constant level of the peptide in human plasma.

### SUMMARY OF THE INVENTION

The above objectives are accomplished according to the present invention by providing in a first embodiment, a novel delivery system for maintaining peptide levels in plasma. The system may include at least one α-CGRP peptide, at least one alginate polymer, wherein the at least one α-CGRP peptide is encapsulated in the at least one alginate polymer to form at least one alginate-α-CGRP peptide. Still yet, the delivery system may release the at least one α-CGRP peptide over time to maintain a constant level of the at least one α-CGRP peptide in plasma. Further, the at least one α-CGRP peptide may remain biologically active after encapsulation. Yet still, the at least one α-CGRP peptide may be encapsulated via an electrospray method. Again, the at least one alginate-α-CGRP peptide remains stable for up to one year at room temperature. Still again, the at least one alginate-α-CGRP peptide may lowers blood pressure. Further again, the system may be tunable to arrive at a pre-selected dosage of the at least one α-CGRP peptide delivered over an extended period of time. Yet further, the at least one alginate polymer may comprise sodium-alginate. Again still, the at least one alginate-α-CGRP peptide may be introduced via subcutaneous administration. Still yet further, herein the at least one α-CGRP peptide may be replaced with at least one α-CGRP peptide agonist analog.

In a further embodiment, a method for forming an alginate-based drug delivery system is provided. The method may include suspending at least one alginate polymer in a liquid, preparing a stock solution of at least one α-CGRP peptide, preparing an ionic gelling bath solution, mixing the at least one alginate polymer and the at least one at least one α-CGRP peptide to form a mixture, flowing the mixture through a charge into the ionic gelling bath solution to encapsulate the at least one α-CGRP peptide in the at least one alginate polymer to form at least one alginate-α-CGRP peptide microcapsule. Still further, the at least one alginate-α-CGRP peptide microcapsule may be formed to be introduced via subcutaneous administration. Yet still, the ionic gelling batch solution may comprise calcium chloride. Further yet, the method may include coating the at least one alginate-α-CGRP peptide microcapsule with at least one amino acid chain. Still yet, the at least one amino acid chain may be poly-L-ornithine or poly-L-lysine. Further still, the at least one alginate-α-CGRP peptide microcapsule may be irradiated with ultraviolet light. Further again, size of the at least one alginate-α-CGRP peptide microcapsule may be adjusted via modifying voltage, flow rate, and/or distance to the gelling bath solution. Further still, the method may include coating the at least one alginate-α-CGRP peptide microcapsule with chitosan.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1A shows a diagram of an alginate-αCGRP microcapsule.

FIG. 1B shows a poly-L-ornithine coated alginate-αCGRP microcapsule.

FIG. 1C shows representative bright field images of alginate-only and alginate-αCGRP microcapsules, scale=200 μm.

FIG. 1D shows the size of prepared alginate-only and alginate-αCGRP microcapsules measured and plotted.

FIG. 2A shows a graph of the release of α-CGRP from an alginate-αCGRP microcapsule.

FIG. 2B shows a graph of the release of α-CGRP from a poly-L-ornithine coated alginate-αCGRP microcapsule.

FIG. 3A shows representative bright contrast images showing the morphology of rat cardiac cell, H9c2 cell, after 7 days treatment with α-CGRP-alone, or alginate-αCGRP microcapsules.

FIG. 3B shows after 7 days of treatments, cells were trypsinized and live cells were counted by trypan blue assay and plotted.

FIG. 3C shows the viability of HL-1 cells in presence of alginate-αCGRP microcapsules as determined by in vitro calcium flux fluorescence assay.

FIG. 4 shows Alginate-αCGRP dose response curve for effect on blood pressure.

FIG. 5 shows at: (A) electrospray method used to encapsulate α-CGRP in alginate polymer; (B) prepared alginate-only and alginate-α-CGRP microcapsules were photographed; (C) measurement and plotting of (B); (D) in vitro α-CGRP release assay showing amount of α-CGRP released in supernatant from alginate-α-CGRP microcapsules; (E) a bar diagram showing number of live H9C2 cells, as measured by trypan-blue cell viability assay; and (F) viability of mouse HL-1 cardiac cells in presence of alginate-α-CGRP microcapsules (10 μM).

FIG. 6 shows at: (A) representative echocardiograms showing short axis B- and M-mode 2D echocardiography performed after 28 days delivery of alginate-α-CGRP; and at (B) and (C) percentage fractional shortening (FS) and ejection fraction (EF) was calculated at various time points and plotted.

FIG. 7 shows at: (A) representative images showing the size of the hearts after 28 days delivery of alginate-α-CGRP microcapsules; (B and C) bar diagrams showing the ratio of wet heart weight/tibia length, and wet lung weight/tibia length; (D) paraffin-embedded LV sections were stained with H&E, WGA stain; (E) stained sections were used to measure cardiomyocyte size in LVs by NIH-ImageJ software and plotted; (F) IN collagen content was quantitated by NIH-ImageJ software and plotted.

FIG. 8 shows at: (A) Western blot showing level of cleaved caspase-3 protein in LVs from sham, sham-alginate-α-CGRP, TAC, and TAC-alginate-α-CGRP; (B) representative fluorescence images showing cleaved caspase-3 staining (green) to detect apoptosis in the LV sections; (C) cleaved caspase-3 positive cells (green) were counted and plotted as the mean±SEM; (D and E) fluorescence images showing 4-HNE staining in the paraffin-embedded LV sections; and (F) bar diagrams showing glutathione (GSH) level in the LVs.

FIG. 9 shows at: (A) a graph showing % FS in sham, sham-alginate-α-CGRP, TAC-only, and TAC-alginate-α-CGRP groups of mice; (B) representative images showing the size of hearts after 28 days delivery of alginate-α-CGRP microcapsules; (C) ratio of wet heart weight/tibia length was plotted as mean±SEM; (D) a bar diagram showing ratio of wet lung weight/tibia length as mean±SEM; (E) a bar diagram showing mice weight gain (in percentage) during the course of experiment as mean±SEM; (F) representative histology images showing size of cardiomyocytes (WGA staining) and level of fibrosis (trichrome-collagen staining) in the LVs from different groups of mice; (G) cardiomyocyte size; and (H) percent fibrosis quantitated using NIH-ImageJ software and plotted.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

### Definitions

4-HNE: 4-hydroxynonenal

α-CGRP: alpha-calcitonin gene-related peptide

A-PLO: alginate-poly-L-ornithine

BP: Blood pressure

CaCl₂: calcium chloride

CVD: cardiovascular diseases

EF: ejection fraction

FS: fractional shortening

GSH: Glutathione

KO: knock-out

LV: left ventricle

S.C.: subcutaneous

TAC: transverse aortic constriction

UV: ultraviolet

WGA: Wheat germ agglutinin

The aim of the present disclosure is to develop novel alginate based drug delivery system applicable to long-term controlled release of α-CGRP in humans. Using electrospray method, the inventors have developed α-CGRP encapsulated alginate microcapsules. Prepared alginate-αCGRP microcapsules release α-CGRP for extended periods of time, and lower blood pressure, as evidenced by mice studies. The animal study also confirms that released α-CGRP from the alginate-αCGRP microcapsules is biologically active. It is also important to note that alginate-αCGRP microcapsules remain stable up to more than one year at room temperature, and do not affect the viability of cardiac cells in in vitro cell culture conditions. Thus, the inventors' novel state-of-the-art technology to encapsulate α-CGRP into alginate polymer and its delivery through alginate microcapsules will be benefit people suffering from cardiovascular diseases.

Alpha-calcitonin gene related peptide (α-CGRP) is a 37-amino acid neuropeptide and is a potent vasodilator. Genetic and pharmacological studies from the inventors' laboratory and others established a protective role of α-CGRP in various cardiovascular diseases including experimental hypertension, heart failure, and myocardial ischemia.

In addition to other studies, the inventors' laboratory demonstrated that absence of α-CGRP gene increased cardiac hypertrophy and dysfunction in pressure-overload induced heart failure in α-CGRP knock-out mice compared to their wild-type counterparts. In recent work, the inventors showed that exogenous administration of α-CGRP, via mini-osmotic pumps for 28 days, protects the heart from transverse aortic constriction pressure-overload induced heart failure in wild-type mice. These studies demonstrated that α-CGRP delivery significantly preserves the heart at functional and anatomical levels by preventing apoptosis, fibrosis, and oxidative stress in pressure-overload mice.

However, non-applicability of osmotic pumps in humans and short half-life of α-CGRP (˜5.5 min in human plasma) limit this approach to use α-CGRP as a drug in humans. To overcome this problem, the inventors developed a novel drug delivery system for α-CGRP in order to maintain a constant level of the peptide in human plasma. The inventors use alginate polymer as a drug carrier and encapsulated native α-CGRP.

The inventors' observed that alginate-αCGRP microcapsules remain stable more than one year at room temperature, and α-CGRP is released from the alginate microcapsules in time-fashion. Alginate-αCGRP microcapsules do not exhibit cellular toxicity when incubated with two different cardiac cell lines, rat H9C2 cells and mouse HL-1 cells. Subcutaneous administration of alginate-αCGRP microcapsules lowers blood pressure in mice indicating that released encapsulated α-CGRP is biologically active in vivo. As an alginate polymer is non-toxic and immunologically inactive, alginate-based drug formulations prepared with α-CGRP peptide will not generate any adverse effects in patients suffering from various cardiovascular diseases, including myocardial infarction, heart failure, and hypertension. The success of this novel drug delivery technology will have the potential to dramatically change conventional drug therapies used presently to treat failing hearts.

The problem with the native peptide is that it lasts in the body for roughly 5-7 minutes. The current disclosure will protect the degradation of the peptide and still allow for the healing effects of the peptide. The capsules are made of a biocompatible FDA approved alginate polymer. The FDA approved polymer delivers the peptide, which is tunable to arrive at the correct dosage of peptide delivered over an extended period of time. The method to create the system is simple and cost effective and can be mass produced.

Material and Methods

Encapsulation of α-CGRP into Alginate Polymer

Sodium-alginate with high mannuronic acid content and low viscosity was purchased from Sigma (St Louise, Mo.). The inventors used an electrospray method to encapsulate native α-CGRP into 2% (w/v) alginate microcapsules. To prepare 2% alginic acid solution, sodium-alginate was suspended in sterile triple distilled water at a concentration of 2% w/v under sterile conditions. The resulting mixture was filtered through 0.2 μm syringe filter. A stock of 2 mg/ml native rat/mouse α-CGRP (GenScript, Piscataway. N.J.) was prepared in sterile saline solution (0.9% sodium chloride, Sigma), and filter sterilized through 0.2 μm syringe filter.

A fresh stock solution of α-CGRP was prepared before each encapsulation experiment. About 250 μl of α-CGRP solution (containing 500 μg of α-CGRP) was mixed with 1 ml of 2% alginic acid solution. Approximately 300 μl of resulting alginate-αCGRP mixture was loaded into a 3cc syringe and attached to a syringe pump. A 50 ml beaker filled with 30 ml of ionic gelling bath solution containing 150 mM calcium chloride (CaCl₂; Sigma) was placed below the syringe pump. The distance between the syringe needle to CaCl₂) gelling bath solution was kept 7 mm.

A high voltage generator was attached to the needle tip, and a constant voltage (6 KV) was set to pass a field of current through the needle tip attached to the syringe. As the alginate-αCGRP mixture was passed through the positively charged syringe needle at a constant rate (flow rate: 60 mm/hr) under high voltage current into the negatively charged CaCl₂ gelling bath, creating spherical Ca⁺² coated alginate-αCGRP microcapsules of 200 μm size. Similar procedures were repeated with remaining 600 μl of alginate-αCGRP mixture. Alginate-only microcapsules were used as a control. Prepared alginate-only and alginate-αCGRP microcapsules were rinsed 4-5 times with sterile triple distilled water for 5 min each to remove excess CaCl₂, and finally suspended in 500 μl of sterile triple distilled water.

The inventors also prepared poly-L-ornithine-coated alginate-αCGRP microcapsules under conditions discussed as above except adding 0.5% poly-L-ornithine in CaCl₂ gelling bath solution. Poly-L-ornithine (PLO) coating was used to increase the integrity of microcapsules. In another embodiment, prepared PLO-coated alginate-αCGRP microcapsules were irradiated with Ultra-violet (UV) light (9999 μJ×100) for 10 min (5 min UV exposure for two times) using a Stratagene UV Stratalinker 1800. Prepared microcapsules were rinsed 4-5 times with sterile triple distilled water for 5 min each, and finally suspended in sterile triple distilled water.

Administration of Alginate-αCGRP Microcapsules

The animal protocols used for this study were in accordance with the guidelines of the National Institutes of Health (NIH), USA, and were approved by the University of South Carolina Institutional Animal Care and Use Committee (USC-IACUC). Eight weeks old C57/BL6 male mice were purchased from Charles River Laboratories, Wilmington, Mass. Mice were housed in the institutional animal facility maintained at 25° C. with an automatic 12 h light/dark cycle, and received a standard diet and tap water with no restrictions. Mice were allowed to acclimate for one week before the start of experiment.

A total 500 μl of alginate-αCGRP microcapsules (containing 150, 250, and 500 μg α-CGRP per 25 g mouse) in sterile 0.9% NaCl saline solution was injected subcutaneously into the flank region of mice using a sterile 27-gauge needle.

Blood Pressure Measurement

Blood pressure of mice was recorded by a non-invasive tail-cuff method using MC4000 Blood Pressure Analysis System (Hatteras Instruments, Cary, N.C.). To reduce stress-induced changes, mice were trained at least three consecutive days prior to baseline blood pressure recording. On the day of blood pressure measurement, mice were normalized in the recording room for at least 1 h, and kept on the instrument platform for 5 min to bring animal body temperature to instrument temperature. After measuring baseline blood pressure (designated as 0 h), 500 μl of alginate-αCGRP microcapsules (containing 150, 250, and 500 μg of α-CGRP) were administered subcutaneously into the flank region of mice and blood pressure was measured at different time points.

Release Profile of α-CGRP from Alginate-αCGRP Microcapsules

The release of α-CGRP from alginate-αCGRP microcapsules was determined using a bicinchoninic acid based MicroBCA protein assay kit (Pierce/ThermoScientific, Waltham, Mass.). Briefly, alginate-αCGRP microcapsules were suspended in 500 μl of sterile triple distilled water and kept at 37° C. The supernatant (250 μl) was collected at various time points, and the volume was made up each time with sterile water. The collected supernatant was stored at 4° C., and released α-CGRP concentration was determined by MicroBCA protein assay kit according to manufacturers instructions (Pierce). Supernatant collected from alginate-only microcapsules was used as a control. Standard curve was prepared with known concentrations of rat/mouse native α-CGRP. Final absorbance was measured at 450 nm in Spectramax Plus-884 microplate reader (Molecular Devices, Sunnyvale, Calif.), and plotted.

Cell Viability Assays

Two different cardiac cell lines, rat H9C2 cells and mouse HL-1 cells, and two different assays, trypan-blue cell viability assay and calcium dye fluorescent based assay, were used to determine the cytotoxicity of prepared alginate-αCGRP microcapsules.

Trypan-blue cell viability assay: Rat cardiac myoblast cell line, H9C2 cells, was cultured in complete culture medium containing Dulbecco's Modified Eagles' Medium (DMEM) supplemented with 10% fetal bovine serum (FBS), 4.5 gm/liter D-glucose, 1.5 gm/liter sodium bicarbonate, and antibiotic solution of 100 unit/ml penicillin and 100 μg/ml streptomycin. Cells were grown at 37° C. in a humidified incubator with 5% CO₂, and sub-cultured before they become confluent. The viability of H9C2 cells in presence or absence of alginate-αCGRP microcapsules was determined by trypan blue assay. Stock solution of rat/mouse native α-CGRP (1 mg/ml) was prepared in sterile 0.9% NaCl saline solution and filter sterilized through 0.2 μm syringe filter. H9C2 cells, grown in complete culture medium (DMEM+1.0% FBS) in 60 mm cell culture dishes, were treated with 1 μM or 5 μM concentration of alginate-αCGRP microcapsules or α-CGRP alone. Cells treated with equal volume of alginate-only microcapsules were used as control. Following treatments, cells were photographed every day (up to 7 days) under phase-contrast microscope to examine the cell morphology. After 7 days of treatment, cells were trypsinized and counted by hemocytometer using trypan-blue exclusion method (Sigma) according to manufacturer's instructions. GraphPad Prism program (GraphPad software, La Jolla, Calif.) was used for statistical analysis.

Calcium dye fluorescent based assay: Mouse cardiac muscle cell line, HL-1 cells, were grown on gelatin/fibronectin ECM mixture coated cell culture plates/flasks in Claycomb Basal Medium (Sigma) supplemented with 10% fetal bovine serum (FBS), 0.1 mM norepinephrine in ascorbic acid, 2 mM L-Glutamine, and 1× penicillin/streptomycin soln. HL-1 cells were maintained at 37° C. in a humidified incubator with 5% CO₂, and cell culture media was exchanged every day.

A calcium dye fluorescent based assay was used to observe the viability (beating phenotype) of HL-1 cells. Briefly, when HL-1 cell confluency reached 100%, 500 μl of 5 μM cell permeable calcium indicator dye Fluo-4AM (Invitrogen) in HEPES-buffered Hanks' solution was added in each well of 24-well culture plate. Cells were incubated at 37° C. for 1 h in a humidified incubator, washed, and 500 μl Hanks' solution was added. Cells were immediately viewed under fluorescent microscope equipped with FITC filter (EVOS FL auto2 microscope, Invitrogen). At 10× objective setting, spontaneous contraction of HL-1 cells was videotaped (considered as 0 hour). A volume of 500 μl Hanks' solution containing 10 μM alginate-αCGRP microcapsules was added and videotaped at every 10 min up to 60 min.

Results

Microencapsulation of α-CGRP Peptide

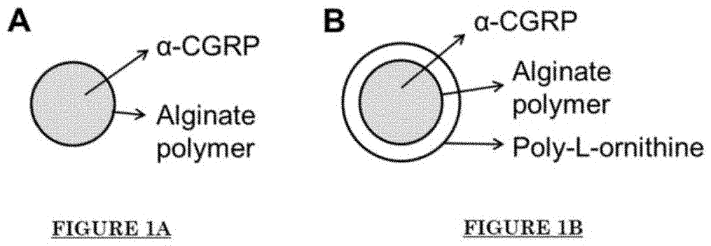

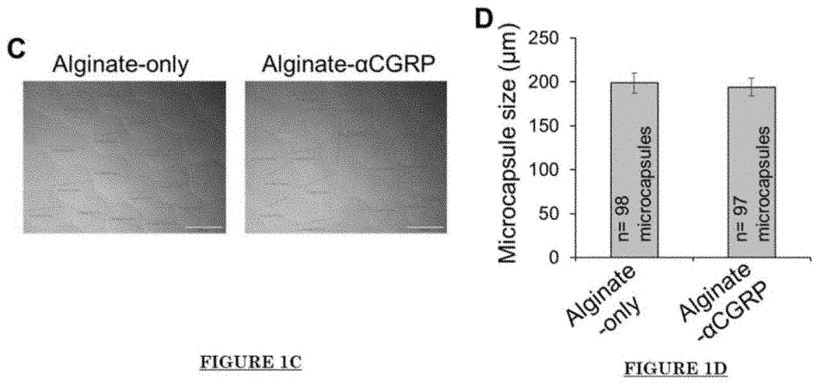

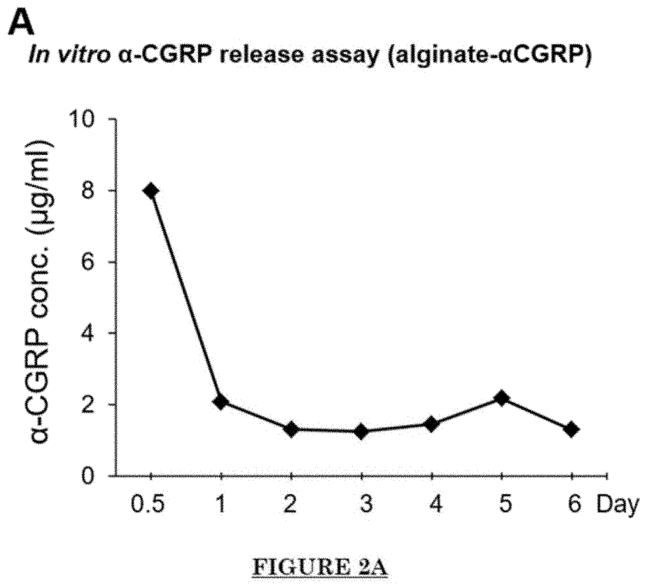

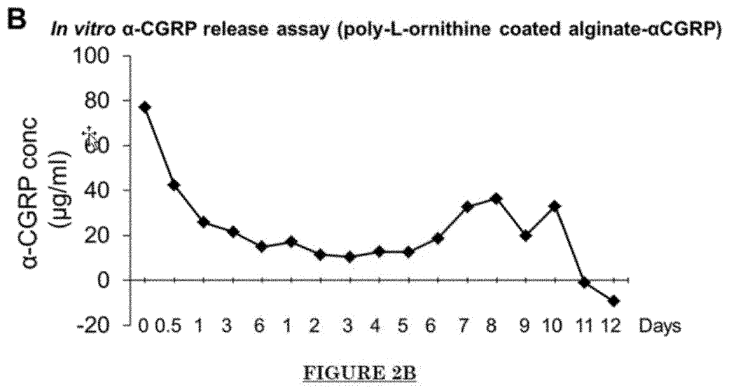

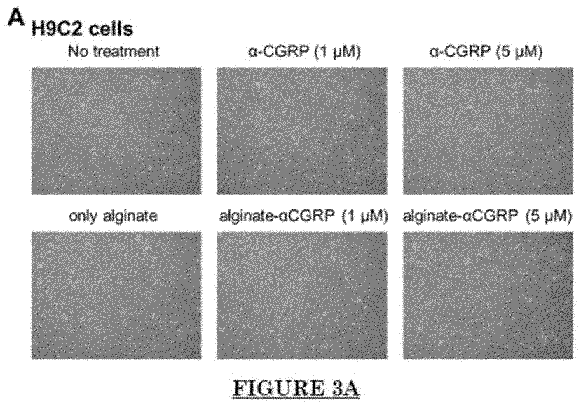

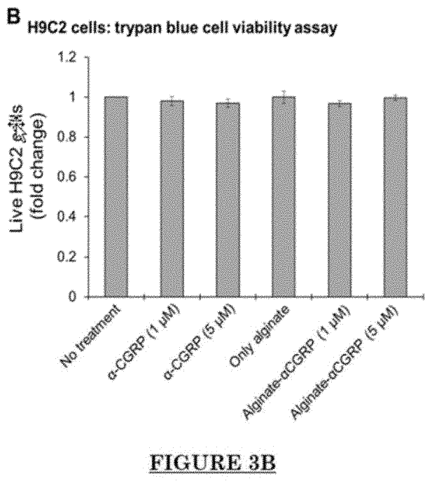

The inventors used electrospray method to encapsulate α-CGRP in alginate polymer. Using extrusion parameters constant at 6.0 kV initial voltage, a flow rate of 60 mm/hr, and distance of syringe needle to CaCl₂) gelling bath solution 7 mm, the inventors prepared alginate-only and alginate-αCGRP microcapsules of 200 μm size (FIGS. 1A-D). A second set of alginate-αCGRP microcapsules of 200 μm size was also prepared containing a second coating of poly-L-ornithine. Prepared poly-L-ornithine coated alginate-αCGRP microcapsules were irradiated with ultraviolet light for 10 min to increase the stiffness of the microcapsules (FIG. 1B). Prepared microcapsules were photographed under Olympus epifluorescence microscope and the size of microcapsules was measured by analysis software included with the microscope (FIGS. 1C and 1D). The calculated average size of alginate-only and alginate-αCGRP microcapsules was 198.84±11.34 μm and 194.23±10.08 μm, respectively (FIG. 2D).

FIGS. 1A-D. Encapsulation of α-CGRP into alginate polymer. Diagram showing alginate-αCGRP microcapsule (A), and poly-L-ornithine coated alginate-αCGRP microcapsule (B). (C) Representative bright field images of alginate-only and alginate-αCGRP microcapsules. Scale=200 sm. The size of prepared alginate-only and alginate-αCGRP microcapsules were measured and plotted (FIG. 1 at D).

FIG. 2. Release profile of α-CGRP peptide from alginate-αCGRP microcapsules. Graphs showing the release of α-CGRP from alginate-αCGRP microcapsule (A), and poly-L-ornithine coated alginate-αCGRP microcapsule (B) at different time points. The concentration of α-CGRP was measured by microBCA protein assay kit using native α-CGRP as a standard.

Release of α-CGRP from Alginate-αCGRP Microcapsules

The release of α-CGRP from the prepared alginate-αCGRP microcapsules (without or with poly-L-ornithine coating) was determined by an in vitro α-CGRP release assay. Alginate-only microcapsules were used as control, and native α-CGRP peptide was used to prepare standard curve. FIG. 2A showed that alginate-αCGRP microcapsules released αCGRP up to 6 days.

Similar to alginate-αCGRP microcapsules, the TV-irradiated poly-L-ornithine coated alginate-αCGRP microcapsules released α-CGRP peptide in to supernatant up to 11 days (FIG. 2B). At later time points, i.e., day 7-day 11, the released αCGRP concentration was higher than the initial time points indicated that some of the microcapsules might get burst at these time points.

Alginate-αCGRP Microcapsules do not Exhibit Cytotoxicity

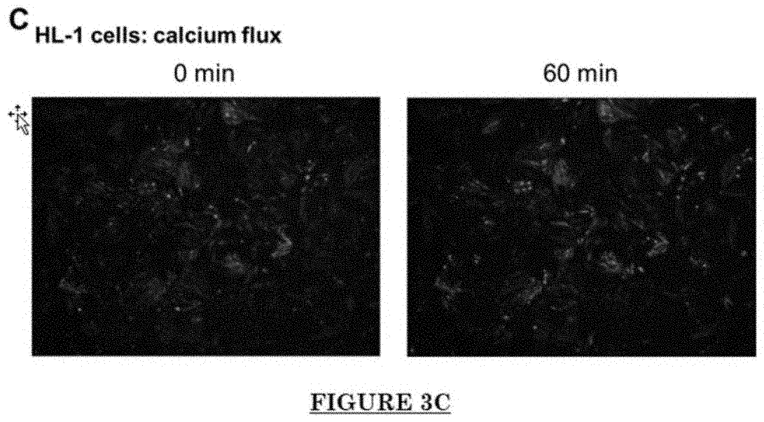

The cellular toxicity of prepared alginate-αCGRP microcapsules was determined by growing rat cardiac cell line-H9C2 cells in the presence of 1 μM and 5 μM of alginate-αCGRP microcapsules. After 7 days of incubation, cells were photographed and trypan blue cell viability assay was carried out. Representative images in FIG. 3A show that the cellular morphology of H9C2 cells in control-untreated, α-CGRP-alone, alginate-only, or alginate-αCGRP microcapsules treated groups was the same (FIG. 3A). Results from trypan blue cell viability assay demonstrated that the viability of H9C2 cells was not significantly different between treatment groups and is comparable to control-untreated cells (FIG. 3B).

FIG. 3. In vitro cell toxicity assay. (A) Representative bright contrast images showing the morphology of rat cardiac cell, H9c2 cell, after 7 days treatment with α-CGRP-alone, alginate-alone, or alginate-αCGRP microcapsules. After 7 days of treatments, cells were trypsinized and live cells were counted by trypan blue assay, and plotted (B). (C) The viability of HL-1 cells in presence of alginate-αCGRP microcapsules was determined by in vitro calcium flux fluorescence assay as discussed in material and method section. HL-1 cells stained with Fluo-4AM dye were videotaped at 0 min, and alginate-αCGRP microcapsules (10 μM) was added. After 60 min incubation, cells were again videotaped (60 min) using EVOS auto F2 microscope.

The viability of HL-1 cell cardiac cells in presence of alginate-αCGRP microcapsules was determined by in vitro calcium flux fluorescence assay as discussed in material and method section. HL-1 cells stained with Fluo-4AM dye were videotaped (to monitor the beating phenotype) and imaged using EVOS auto F2 microscope (considered as time point 0 min). Alginate-αCGRP microcapsules (10 μM) were added in similar well, cells were further videotaped and imaged at various time points. The images (FIG. 3C) and videos (data not shown) taken at time points 0 min 10 and 60 min after alginate-αCGRP microcapsules addition demonstrated that alginate-αCGRP microcapsules (10 μM) did not affect the contractions of HL-1 cells. These results suggest that alginate-αCGRP microcapsules do not exhibit cytotoxicity against cardiac cell lines.

Alginate-αCGRP Microcapsules Reduces Blood Pressure in Mice

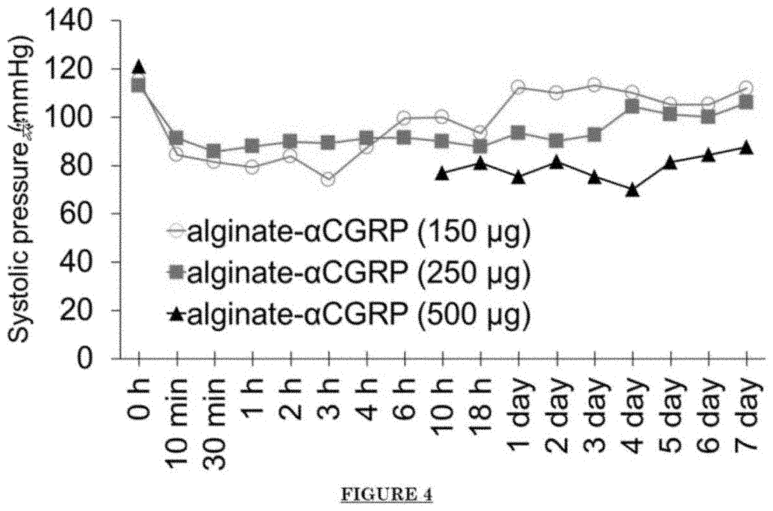

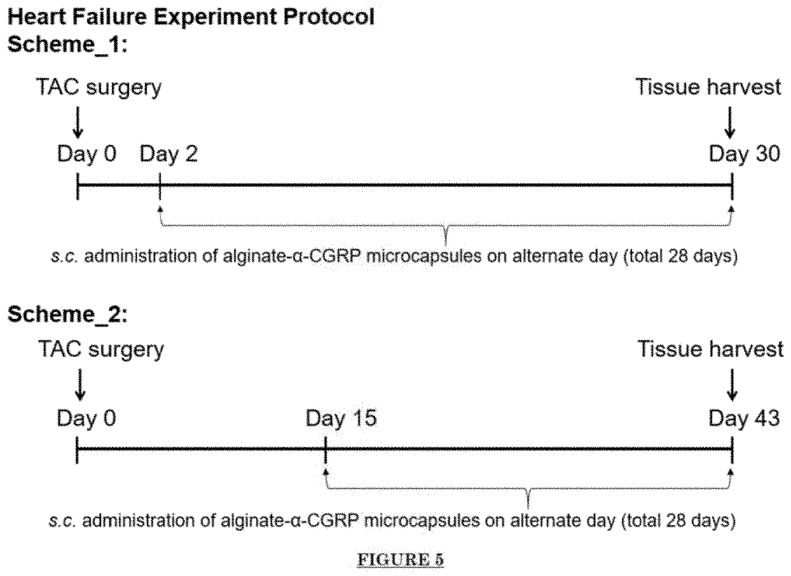

Peptide α-CGRP is a potent vasodilator and is known to reduce blood pressure in normotensive and hypertensive animals and human (DiPette et al, 1989; Dubois-Rande et al, 1.992). Hence a pilot study was conducted in mice to confirm the biological activity of released α-CGRP from alginate-αCGRP microcapsules by measuring blood pressure. Three different doses of alginate microcapsules containing 150 jpg, 250 μg, or 500 μg α-CGRP per 25 g mouse were injected subcutaneously in mice (2 mice/dose) and systolic pressure was monitored by tail-cuff blood pressure. Data shown in FIG. 4 demonstrated that administration of 150 μg and 250 μg alginate-αCGRP microcapsule lowered the systolic pressure up to 18 h and 3 days, respectively, afterward blood pressure returned to the normal basal level. However, subcutaneous administration of 500 μg alginate-αCGRP microcapsules drastically reduced the blood pressure in first 6 h and could not be recognized by the instrument. The blood pressure remained low over 7 days. Nevertheless, subcutaneous administration of equal amount of alginate-only microcapsules did not affect blood pressure in mice. These results confirm that alginate microcapsules release αCGRP under in vivo conditions for an extended period of time, as evidenced by the reduced blood pressure in vivo in the test subject mice.

FIG. 4. Alginate-αCGRP dose response curve (effect on blood pressure). The dose response curve showing the effects of subcutaneously administered different concentrations of alginate-αCGRP microcapsules on systolic blood pressure (mmHg) in the mice (n=2 mice per group). The blood pressure was measured by tail-cuff method.

In the present disclosure, the inventors used alginate polymer as a drug carrier and formed novel alginate-αCGRP microcapsules for the delivery of α-CGRP peptide in humans. The major findings of the present study are: (i)—Prepared alginate-αCGRP microcapsules and UV-irradiated poly-L-ornithine-coated alginate-αCGRP microcapsules release encapsulated α-CGRP for extended period of time in in vitro conditions as well as in vivo in mice, (ii)—Alginate-αCGRP microcapsules do not exhibit cellular toxicity against cardiac cells, and (iii)—Encapsulated α-CGRP is biologically active, as released α-CGRP from alginate-αCGRP microcapsules lowers the blood pressure in wild-type mice.

Alginate is a natural polysaccharide and has been extensively used to encapsulate a wide variety of molecules ranging from large macromolecules, such as cells, DNA and protein, to small molecules-peptides and antibodies. (Lee & Mooney, 2012; Moore et al, 2014). Studies from the inventors' laboratory and others confirmed the protective role of αCGRP in various cardiovascular diseases (Bowers et al, 2005; Li (t al, 2013b; Supowit et al, 2005), and the inventors' recent findings further showed that exogenous delivery of native αCGRP peptide, through mini-osmotic pumps, protects heart against pressure-induced heart failure (Kumar et al, 2019).

However, the short half-life of α-CGRP in human plasma (t_{1/2}=˜5.5 min) makes it difficult to use α-CGRP as a therapeutic agent to treat and prevent cardiac disease. To address this problem, the inventors developed a novel alginate based α-CGRP delivery system in order to deliver peptide in controlled and sustained manner. The inventors' state-of-art technology using electrospray method develops α-CGRP encapsulated alginate microcapsules of 200 μm of size (FIG. 1). The advantage of an electrospray method is that alginate-αCGRP capsules from nano- to micro-size (ranging from 10 nm-500 μm) can be prepared after adjusting the experimental parameters, e.g., the voltage, flow rate, and distance between needle to gelling bath solution. Alginate microcapsules/nanocapsules can also be used to encapsulate αCGRP-agonist analogue derivatives.

Prepared alginate-αCGRP microcapsules/nanocapsules can be further coated with poly-L-ornithine, poly-IL-lysine, and chitosan by adding respective chemical in the gelling bath solution. The coating of poly-L-ornithine, poly-L-lysine, and chitosan might be single-layered or double-layered. The encapsulated micro- or nano-capsules can be further irradiated with ultra-violet light to increase the stiffness of the capsules that further extend the release of α-CGRP peptide. In the present study, the inventors prepared UV-irradiated poly-L-ornithine-coated alginate-αCGRP microcapsules of 200 μm of size (FIG. 1B).

Encapsulated microcapsules are very stable at room temperature as the shape of alginate-alone and alginate-αCGRP microcapsules in deionized water remained intact even after 15 months. The inventors' α-CGRP encapsulation method did not affect the biological activity of α-CGRP as released α-CGRP from subcutaneously administered alginate-αCGRP microcapsules lowers the blood pressure, an inherent property of native αCGRP, in mice (FIG. 4). Two different assays, Trypan blue cell viability assay and in vitro calcium fluorescence assay, were performed with two different cardiac cell lines (rat H9C2 cells and mouse HL-1 cells) to confirm the non-toxic nature of alginate microcapsules (FIG. 3). Alginate-αCGRP microcapsules did not affect the growth of H9C2 cells (as determined by Trypan blue cell exclusion assay, FIG. 3B. Similarly, HL-1 cells keeps beating on the plate even after 1 h incubation with alginate-αCGRP microsphere (FIG. 3C). These in vitro data indicate that alginate-αCGRP microcapsules neither affect the viability nor beating phenotype of cardiac cells.

Several lines of evidence demonstrated that systemic administration of α-CGRP reduces the blood pressure in normal and hypertensive animals and humans, however, the reduction in blood pressure is very short period of time because the half-life of native α-CGRP in human plasma is only 5.5 min (Ando et al. 1990; DiPette et al, 1989; Dubois-Rande et al, 1992; Siren & Feuerstein, 1988). Katsuyuki et. al. (1990) reported that intravenous injections of α-CGRP decreased mean arterial pressure (MAP) significantly in a dose-related fashion in both normal as well as spontaneously hypertensive rats, however MAP returned to normal baseline after 20 min of injection in both groups of rats (Ando et al. 1990). In contrast, the inventors' animal study shows that subcutaneous administration of 150 μg and 250 μg alginate-αCGRP microcapsules lower the systolic pressure up to 18 h and 3 days, respectively, in mice (FIG. 4). The inventors' results suggest that addition of alginate polymer extends the release of peptide, and released α-CGRP remains biologically active in mice.

The inventors' studies demonstrated that alginate-αCGRP microcapsules are stable at room temperature, and releases the peptide in a controlled manner. Alginate polymer is non-toxic and immunologically inactive, hence a prepared alginate based drug formulation (alginate microcapsules/nanocapsules encapsulated with α-CGRP or α-CGRP-agonist analogue) will likely not elicit side effects in humans. The inventors' laboratory reported that alginate microcapsules can undergo freeze-thaw cycles as well as being lyophilized without compromising the integrity of microcapsules. Lyophilized powder form of alginate microcapsules swell and regain their shape when suspended in distilled water. Thus, alginate based drug formulation alginate-αCGRP microcapsules/nanocapsules, in lyophilized powder form and in liquid suspension, can be stored at normal room temperature to very low temperature (below 0° C.), for easy transport.

The prepared alginate-αCGRP drug formulation containing α-CGRP or α-CGRP-agonist analogues can be maintained as a solid, liquid or aerosol form and can be administered to patients by several means such as, but not limited to, by intravenously, subcutaneously, intraperitoneally, intramuscular, intraarterial, topical, transdermal, intravaginal, intrauterine, intraspinal, intracerebral, intracerebroventricular, intracranial, rectal, and through nasal and oral route. The sustained release of αCGRP peptide from alginate-αCGRP microcapsules can also be achieved by mixing with pluronic acid gel solution.

The possible solid compositions (alginate microcapsules/nanocapsules encapsulated with α-CGRP or α-CGRP-agonist analogues) can include, but not limited to, pills, tablets, capsules, solution or elixir, creams, and implantable dosage units. An implantable dosage unit, in the form of patch or mechanical device, can be implanted on the skin or can be administered locally inside the patients' body, for example at a cardiac, kidney or artery site, for systemic release of α-CGRP or α-CGRP-agonist analogues. The possible liquid drug formulations (alginate microcapsules/nanocapsules encapsulated with α-CGRP or α-CGRP-agonist analogues) can be adapted for injection subcutaneously, intravenously, intramuscular, intraarterial, intraocular and transdermal. Possible examples of aerosol formulations for alginate microcapsules/nanocapsules encapsulated with α-CGRP or α-CGRP-agonist analogues may be in inhaler form for direct administration to the lungs.

In addition, alginate microcapsules/nanocapsules encapsulated with α-CGRP or α-CGRP-agonist analogues can be administered alone or in conjunction with other forms of therapy, e.g., and without limitation, chemotherapy, immunotherapy, and surgical intervention in treatment and prevention of cardiovascular diseases.

Overall, alginate microcapsules/nanocapsules based delivery systems have the potential to improve α-CGRP bioavailability in plasma, and increase the duration of the therapeutic effect of the peptide throughout the treatment period. Thus, alginate-αCGRP microcapsules/nanocapsules (with or without coating of poly-L-ornithine, poly-L-lysine, and chitosan, and with and without UV-exposure) are an effective way for controlled and sustained delivery of α-CGRP and α-CGRP-agonist analogue derivatives in humans suffering from various cardiovascular diseases including, but not limited to, cardiac hypertrophy, stroke, dilated cardiomyopathy, idiopathic dilated cardiomyopathy, inherited cardiomyopathy, diabetic-cardiomyopathy, cardiomyopathy induced by chemotherapy (such as doxorubicin) or toxins, myocardial infarction, heart failure (induced by pressure- and volume-overload), cardiac ischemia, and hypertension induced heart failure and kidney damage, and cardiac remodeling induced during pregnancy.

Experimental: In Vivo Heart Failure Study in Mouse Model

The peptide has been encapsulated and cells treated with the peptide to determine toxicity. None was found. The encapsulated peptide was injected into mice and the proper hypotensive response was achieved.

Rationale—α-CGRP (alpha-calcitonin gene related peptide), a potent vasodilator neuropeptide, has been shown in studies from our laboratory and others to have a protective function in a variety of cardiovascular diseases, including heart failure, myocardial infarction, and experimental hypertension. Our recent study demonstrated that exogenous administration of native α-CGRP using osmotic mini-pumps protected the heart from pressure-induced heart failure in wild-type mice. However, the short half-life of peptide and non-applicability of osmotic pumps in human limits the use of α-CGRP as a therapeutic agent for heart failure.

Objective—We sought to comprehensively study a novel α-CGRP delivery system to determine its bioavailability in vivo and test the cardioprotective effect and for the first time treatment of alginate-α-CGRP microcapsules in a mouse model of pressure-overload induced heart failure.

Methods and Result—Native α-CGRP filled alginate microcapsules (200 micron) were prepared using an electrospray method. Mice were divided into four groups: sham, sham-alginate-α-CGRP, TAC-only, and TAC-alginate-α-CGRP, and transaortic constriction (TAC) procedure was performed in TAC-only and TAC-alginate-α-CGRP groups of mice to induce pressure-overload heart failure. After two-day or fifteen-day post-TAC, alginate-α-CGRP microcapsules (containing 150 μg α-CGRP; final α-CGRP dose 6 mg/kg/mouse) were administered subcutaneously on alternate day, for 28 days, and cardiac functions were evaluated by echocardiography weekly. After 28 days of peptide delivery, all groups of mice were sacrificed, hearts were collected, and biochemical and histological analyses were performed. Our data demonstrated for the first time that administration of alginate-α-CGRP microcapsules significantly improved all cardiac parameters examined in TAC mice. When compared to sham mice. TAC markedly increased heart and lung weight, left ventricle (LV) cardiac cell size, cardiac apoptosis and oxidative stress. In contrast, administration of alginate-α-CGRP microcapsules significantly attenuated the increased heart and lung weight, LV cardiomyocytes size, apoptosis and oxidative stress in TAC mice. Finally, we show that administration of alginate-α-CGRP microcapsules just prior to the onset of symptoms has the ability to reverse the deleterious parameters seen in TAC mice.

Our results demonstrate that encapsulation of α-CGRP in alginate polymer is an effective strategy to improve peptide bioavailability in plasma and increase the duration of the therapeutic effect of the peptide throughout the treatment period. Furthermore, alginate mediated α-CGRP delivery, either prior to onset or after initiation of symptom progression of pressure-overload, improves cardiac functions and protects hearts against pressure-overload induced heart failure.

Alpha-calcitonin gene related peptide (α-CGRP), a 37 amino acid neuropeptide, is considered the most potent vasodilator discovered to date, and possesses positive chronotropic and inotropic effects. Extensive studies from our laboratory and others established a protective function for α-CGRP in a variety of cardiovascular diseases, including heart failure, myocardial infarction, and experimental hypertension. ENREF 17 In addition, α-CGRP delivery lowers blood pressure (BP) in normal as well as hypertensive animals and humans. Using α-CGRP knock-out (KO) mice, our laboratory showed that, in comparison with wild-type mice. KO mice exhibited greater cardiac hypertrophy, and cardiac dilation and dysfunction, cardiac fibrosis, and mortality when subjected to transverse aortic constriction (TAC) pressure-overload induced heart failure. Our recent study demonstrated that long-term exogenous delivery of native α-CGRP, through osmotic mini-pumps, attenuated the adverse effects of TAC pressure-overload induced heart failure in wild-type mice. Long term administration of native α-CGRP preserved cardiac function, and reduced apoptotic cell death, fibrosis, and oxidative stress in TAC left ventricles (LVs), thus confirming the cardioprotective function of α-CGRP in congestive heart failure. Similarly, two other studies confirmed that infusion of either native α-CGRP or an α-CGRP-agonist analog (an acylated form of α-CGRP with half-life, t_{1/2}=˜7 h) significantly improved cardiac functions in rodent models of hypertension and heart failure. These lines of evidence further confirm that α-CGRP, either native or its derivative, is a promising drug candidate to treat cardiovascular diseases. However, the short half-life of α-CGRP (t_{1/2}=˜5.5 min in human plasma) and non-applicability of implanted osmotic pumps in humans limits the use of α-CGRP as a therapeutic agent for long-term treatment. Therefore, novel delivery systems are needed that could increase the bioavailability of the peptide in the serum.

Alginate polymers have garnered favor recently as a FDA approved novel drug carrier. This is underscored by several clinical trials on alginate-based drug delivery formulations that are currently ongoing. Alginate is a water soluble linear polysaccharide isolated from the brown algae. Structurally, it is an unbranched polyanionic polysaccharides of 1-4 linked α-L-guluronic acid and 6-D)-mannuronic acid. As the alginate polymer in stable at wide range of temperature (0-1.00° C.), non-toxic, and biocompatible, a variety of biomolecules ranging from peptides, DNA, antibodies, proteins to cells have been used for encapsulation. Our laboratory has routinely utilized alginate-based drug delivery technology to encapsulate various proteins, inhibitors, and cells, to treat both corneal wounds in diabetic rate and macular degeneration in a mouse model.

The aim of the present disclosure was to develop a novel alginate based drug delivery system applicable of long-term sustained release of α-CGRP in humans. We used an electrospray method to encapsulate α-CGRP in alginate microcapsules and tested its efficacy in TAC pressure-overload induced heart failure both as a prevention and treatment. Our results show that subcutaneous administration of alginate-α-CGRP microcapsules immediately after TAC surgery and prior to the onset of symptoms significantly protects hearts at the physiological and cellular level. Thus, our novel state-of-the-art technology to encapsulate α-CGRP and its delivery through alginate microcapsules offers new options to benefit people suffering from cardiovascular diseases.

Methods

Preparation of Alginate-α-CGRP Microcapsules

An electrospray method was used to prepare α-CGRP encapsulated alginate microcapsules of 200 μm size. Briefly, 2% alginic acid solution (high mannuronic acid content and low viscosity; MilliporeSigma, St. Louis, Mo.) was prepared in sterile triple distilled water and filtered through 0.2 μm syringe filter. A stock solution of 2 mg/ml of rat/mouse native α-CGRP (GenScript USA Inc. Piscataway, N.J.) was prepared in sterile 0.9% NaCl saline solution and further filter sterilized through 2 μm syringe filter. Five hundred microgram of prepared α-CGRP was mixed with 1 ml of 2% alginic acid and passed through positively charged syringe at a constant rate under high voltage current into the 150 mM CaCl₂ gelling solution to make calcium-coated alginate-α-CGRP microcapsules. Prepared microcapsules were washed 4-5 times with sterile triple distilled water for 5 min each to remove excess CaCl₂ and α-CGRP filled microcapsules were finally suspended in 500 μl of sterile triple distilled water. Alginate-only microcapsules were prepared under similar conditions. Release of peptide from alginate-α-CGRP microcapsules was confirmed by in vitro α-CGRP release assay. Briefly, 250 μl supernatant was collected at various time points and stored at 4° C. and the volume was made up each time with sterile water. Peptide concentration in the supernatant was quantitated by MicroBCA protein assay kit (Pierce/ThermoScientific, Waltham, Mass.) using rat/mouse α-CGRP as standard. Supernatant collected from alginate-only microcapsules was used as control. Final absorbance was measured at 450 nm using Spectramax Plus-384 microplate reader (Molecular Devices, Sunnyvale, Calif.) and plotted.

Pressure-Overload Heart Failure Mouse Model

Eight-week-old male C57/BL6 mice (Charles River Laboratories, Wilmington, Mass.) were maintained on a 12 h light/12 h dark cycle with free access to standard food and water. Mice were allowed to acclimate for one week after shipment. The animal protocols were approved by the University of South Carolina-Institutional Animal Care and Use Committee following the National Institutes of Health (NIH), USA, guidelines.

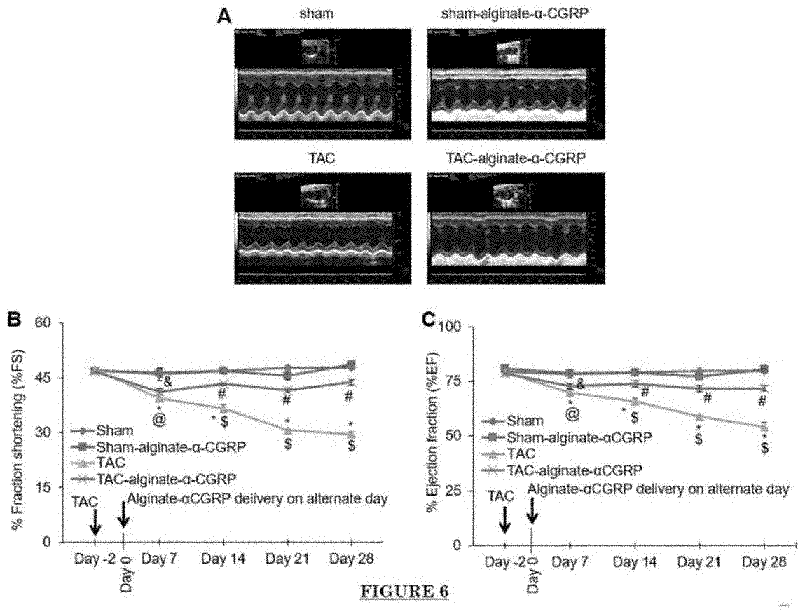

Transverse aortic constriction (TAC) procedure in mice was performed to induce pressure-overload heart failure. Briefly, chest of anesthetized mice (under 1-1.5% isoflurane) was opened through the suprasternal notch, and 7-0 suture (Ethicon prolene polypropylene blue) was passed under the aortic arch between the left common carotid and innominate arteries. The suture was tied around both the aorta and a 27-gauge needle. After placing a knot, the needle was removed. This procedure yield 70.80% aortic constriction. The chest was closed using 6-0 silk suture and mice were allowed to recover. Sham-operated mice underwent an identical procedure except for the aortic constriction. Two days post-surgery, mice were divided into four groups: sham (n=8), sham-alginate-CGRP (n=7), TAC-only (n=7), and TAC-alginate-CGRP (n=8). In the sham-alginate-CGRP and TAC-alginate-CGRP groups of mice, α-CGRP-encapsulated alginate microcapsules (containing 150 μg of α-CGRP; final α-CGRP dose 6 mg/kg/mouse) were injected subcutaneously into the flank region of mice on alternate day, for 28 days. At the end of the experiment (day 28 of α-CGRP delivery), mice from all groups were weighed and euthanized. The wet weight of hearts and lungs were measured and photographed. Basal portion of the heart left ventricle (LV) was fixed in 4% paraformaldehyde/PBS (pH 7.4) for histochemistry, while apical portion was snap frozen in liquid N₂ and stored at −80° C. for biochemical analyses. In addition, the treatment protocol was performed for α-CGRP in which mice were divided in to four groups: sham (n=5), sham-alginate-CGRP (n=4), TAC-only (n=4), and TAC-alginate-CGRP (n=4), and fifteen-day post-TAC, alginate-α-CGRP microcapsules (containing 150 μg of α-CGRP; final α-CGRP dose 6 mg/kg/mouse) were injected subcutaneously into the flank region of mice on alternate day, for 28 days. The treatment regime for both studies is found in supplemental data, see FIG. 5. At the conclusion of the study (day 28), mice were euthanized, and tissues were collected as discussed before.

Transthoracic Echocardiography

A Vevo 3100 High-Resolution Imaging System (VisualSonics Inc, Toronto, Canada) was used to perform echocardiography in mice. Briefly, mice were sedated under 2% isoflurane and mice heart rate was maintained at 450*20 beats per minute. Short axis B- and M-mode 2D echocardiograms were recorded through the anterior and posterior LV walls at the level of the papillary muscle. Fractional shortening (FS) and ejection fraction (EF) were calculated by the VisualSonics Measurement Software.

Blood Pressure Measurement

Blood pressure (BP1) of sham and treatment mice was recorded by non-invasive tail-cuff method using MC4000 BP Analysis System (Hatteras Instruments, Cary, N.C.). To reduce stress-induced changes, mice were trained at least three-to-five consecutive days prior to baseline BP recording. On the day of BP measurement, mice were normalized in the recording room for at least 1 h, and kept on the instrument platform for 5 min to bring animal body temperature to the instrument temperature. After measuring baseline BP (designated as 0 h), alginate microcapsules (with or without α-CGRP) were administered subcutaneously into the flank region of mice and BP was again recorded at various time points.

Western Blotting

Total protein from the LVs was extracted using RIPA cell lysis buffer (Cell Signaling Technology, Danvers, Mass.), and protein concentration was measured by BCA protein assay kit (Pierce). Equal amount of protein samples (40 μg) were mixed with 5× Laemmli sample buffer, heated at 95° C. for 10 min, and separated on SDS-polyacrylamide gel followed by transfer on PVDF membrane at 100 volt for 3 h in the cold room. Membrane was blocked with 10% non-fat dry milk prepared in TBST (20 mM Tris-Cl, pH 7.4; 150 mM NaCl with 0.1% Tween-20) for 4 h at room temperature and further incubated in primary antibodies for overnight at 4° C. Protein signals were detected by adding HRP-conjugated secondary antibodies (Bio-Rad Laboratories, Hercules, Calif.) for 2 h at room temperature and using Clarity Western Detection Kit (Bio-Rad). Primary antibodies used were cleaved caspase-3 and 6-actin (Cell Signaling Technology).

Immunohistochemistry

Paraformaldehyde-fixed paraffin-embedded LV sections (5 μm) were deparaffinized and rehydrated with xylene and graded ethanol (100%, 95%, and 70%), respectively, and boiled in 10 mM sodium citrate buffer (pH 6.0) for 30 min for antigen retrieval. After permeabilization with 0.2% Triton X-100/PBS for 10 min, LV sections were blocked with 10% IgG-free-BSA/PBS (Jackson ImmunoResearch Laboratories, West Grove, Pa.) and incubated with primary antibodies for overnight at 4° C. Alexafluor-488 or Alexafluor-546 conjugated secondary antibodies (Invitrogen, Carlsbad, Calif.) were added to detect protein signals. After mounting with antifade-mounting media (Vector Laboratories, Burlingame, Calif.), tissue sections were examined under Nikon-E600 fluorescence microscope (Nikon, Japan). Primary antibodies used were: cleaved caspase-3 (Cell Signaling) and anti-4-hydroxy-2-nonenal (4-HNE; Abcam Inc, Cambridge, Mass.). DAPI (4′,6-diamidino-2-phenylindole; Sigma) was used to stain nuclei.

Hematoxylin and Eosin (H&E) staining, Texas Red-X conjugated wheat germ agglutinin staining (WGA staining; Invitrogen) and Masson's trichrome-collagen staining (PolyScientific, Bay Shore, N.Y.) were performed using vendors' protocol to measure LV cardiac cell size, cardiomyocyte cross-sectional area, and fibrosis, respectively, and quantitated using NIH-ImageJ software (NIH, USA).

Cardiac Cell Lines and In Vitro Cytotoxicity Assays

Trypan-blue cell viability assay: The rat cardiac H9C2 cells were grown at 37° C. in a humidified incubator with 5% CO₂ in complete culture medium (containing DMEM supplemented with 10% fetal bovine serum, FBS, 4.5 gm/liter D-glucose, and 1× penicillin/streptomycin). The viability of H9C2 cells in presence of alginate-α-CGRP microcapsules was determined by trypan-blue assay (Sigma). Briefly, stock solution of rat/mouse α-CGRP (1 mg/ml) was prepared in sterile 0.9% NaCl solution and filter sterilized through 0.2 μm syringe filter. H9C2 cells, grown in complete culture medium, were treated with alginate-only, α-CGRP, or alginate-α-CGRP microcapsules. Following treatments, cells were photographed under phase-contrast microscope to examine the cell morphology. After 7 days of treatment, cells were trypsinized and counted by hemocytometer using trypan-blue exclusion method.

Calcium dye fluorescent based assay: The mouse cardiac muscle cell line, HL-1 cells, were grown on gelatin and fibronectin-coated cell culture flasks in Claycomb Basal Medium (Sigma) supplemented with 10% FBS, 0.1 mM norepinephrine in ascorbic acid, 2 mM L-glutamine, and 1× penicillin/streptomycin soln. HL-1 cells were maintained at 37° C. in a humidified incubator with 5% CO₂, and cell culture media was exchanged on every day.

A cell permeant calcium dye fluorescent based assay was performed in gelatin and fibronectin-coated 24-well culture plate to observe the viability (beating phenotype) of HL-1 cells. Briefly, at 100% cell confluency, 500 μl of 5 μM cell permeable calcium indicator dye Fluo-4AM (Invitrogen) in HEPES-buffered Hanks' solution was added in each well followed by incubation at 37° C. for 1 h in a humidified incubator. After incubation, cells were washed in Hanks' solution and 500 yl Hanks' solution was added. Cells were immediately viewed using the EVOS FL auto2 microscope (Invitrogen). Using the 10× objective setting, spontaneous contraction of HL-1 cells was video recorded (considered as 0 hour). A volume of 500 μl Hanks' solution containing 10 μM alginate-α-CGRP microcapsules was added and video recorded at every 10 min for 60 min.

Enzymatic Activity Assay

GSH-Glo Glutathione assay kit (Promega) was used to measure total glutathione (GSH) content in the LVs following vendor's instructions. Briefly, 10 mg LV heart tissue was homogenized in 1×PBS containing 2 mM EDTA, centrifuged at 12,000 rpm for 15 min at 4° C., and supernatant was collected. 50 μl of GSH-Glo Reagent was mixed with 50 μl of tissue extract (10 jpg) and incubated for 30 min at RT. Next, 100 μl of luciferin detection reagent was added and incubated for an additional 15 min at RT. The signal was measured using a Turner 20/20 luminometer (Promega).

Statistical Analysis

Comparisons were made among the groups using student t-test and one-way ANOVA followed by Tukey-Kramer ad hoc test (GraphPad software, La Jolla, Calif.), p value<0.05 was considered significant.

Results

Encapsulation of α-CGRP and release from alginate microcapsules

α-CGRP was encapsulated using an electrospray method with following experimental conditions to prepare 200 μm size alginate-α-CGRP microcapsules. α-CGRP (500 μg from a stock 2 mg/ml soln) was mixed with 1 ml of 2% alginic acid solution and loaded to 3 ml syringe attached with high-voltage generator. A beaker filled with 30 ml of ionic gelling bath solution containing 150 mM CaCl₂ was placed below the syringe pump and the distance between the syringe needle to CaCl₂ gelling bath solution was kept 7 mm. As the alginate-α-CGRP mixture was passed through the positively charged syringe needle at a constant rate (flow rate: 60 mm/hr) under high voltage current (6 KV) into the negatively charged CaCl₂) gelling bath, creating spherical Ca⁺²-coated alginate-α-CGRP microcapsules of 200 μm size. We also prepared alginate-only microcapsules of similar size. Prepared microcapsules were photographed and the size of microcapsules was measured. The calculated average size of alginate-only and alginate-α-CGRP microcapsules was 198.84±11.34 μm and 194.23±10.08 μm, respectively (FIG. 5 at A-C). Release of α-CGRP from the prepared alginate-α-CGRP microcapsules was determined by an in vitro α-CGRP release assay. FIG. 5 at D showed that presence of α-CGRP was detected in the supernatant for up to 6 days indicating that alginate-α-CGRP microcapsules released peptide over an extended period of time.

Alginate-α-CGRP Microcapsules Exhibit No Cytotoxicity

It is crucial in determining the effect of the release of α-CGRP on the heart to show that cardiac muscle cells are not altered by the addition of the capsules. To that end we used two different cardiac cell lines-rat H9C2 cells and mouse HL-1 cells, and two different cell viability assays-trypan-blue exclusion assay and calcium dye fluorescent based assay, to determine the cytotoxicity of prepared alginate-α-CGRP microcapsules. H9C2 cells were grown in complete culture medium in presence of 1 μM or 5 μM of alginate-α-CGRP microcapsules. After 7 days of incubation with the capsules, a trypan-blue exclusion assay was carried out. Results from the assay demonstrated that the viability of H9C2 cells was similar among the treatment groups when compared to control-untreated cells (ns=non-significant compared to control, see FIG. 5 at E.

The viability of mouse HL-1 cardiac cells in presence of alginate-α-CGRP microcapsules was determined using an in vitro calcium flux fluorescence assay. HL-1 cells stained with Fluo-4AM dye were video recorded to monitor both the beating phenotype and calcium fluxes inside the cell and imaged using an EVOS auto-F2 microscope. After taking images at basal time point (0 min), alginate-α-CGRP microcapsules (10 μM) were added and were further video recorded. Images, see FIG. 5 at F) taken at time points 0 min and 60 min after addition of alginate-α-CGRP microcapsules demonstrated that the alginate-α-CGRP microcapsules (10 μM) did not affect the myocyte contraction of HL-1 cells. These data support our statement that alginate-α-CGRP microcapsules do not exhibit cytotoxicity against the cardiac cell lines tested.

Alginate-α-CGRP Microcapsules Delivery Improves Cardiac Functions in TAC Mice

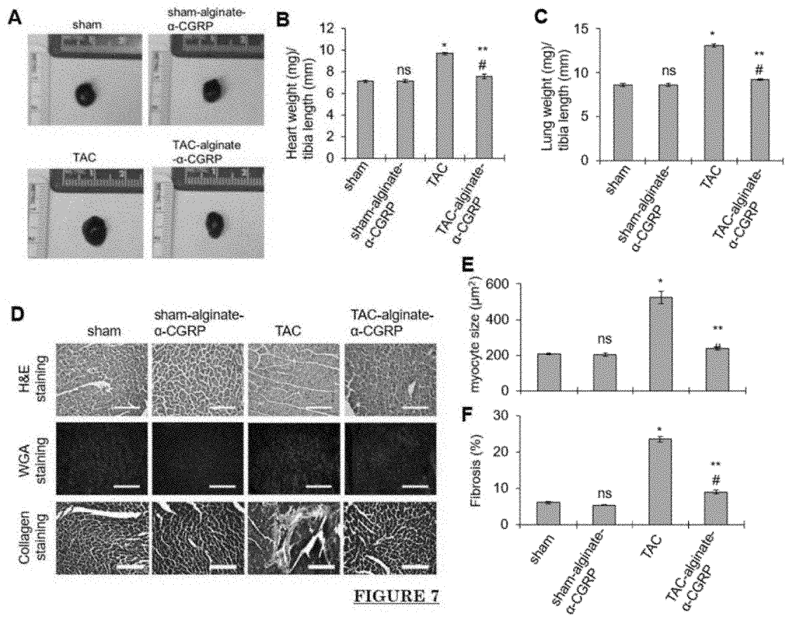

Our previous studies demonstrated that continual α-CGRP administration following TAC surgery showed a cardioprotective capability. Therefore to determine if the alginate-α-CGRP microcapsules also had a cardioprotective effect, B- and M-mode 2D electrocardiography was performed on every 7^{th} day, up to day 28, following subcutaneous administration of 150 μg alginate-α-CGRP microcapsules; final α-CGRP dose 6 mg/kg/mouse, FIG. 6 at A-C. Over the course of experiment, LV systolic function was assessed by measuring both % fraction shortening, see FIG. 6 at B, and ejection fraction, see FIG. 6 at C. Both measures were significantly decreased as expected in the TAC mice when compared to the sham mice. However, repeated administration of alginate-α-CGRP microcapsules starting 2 days after TAC surgery showed significant preservation of both cardiac parameters in treated TAC mice.

α-CGRP Administration Attenuates Cardiac Hypertrophy and Fibrosis in TAC Mice

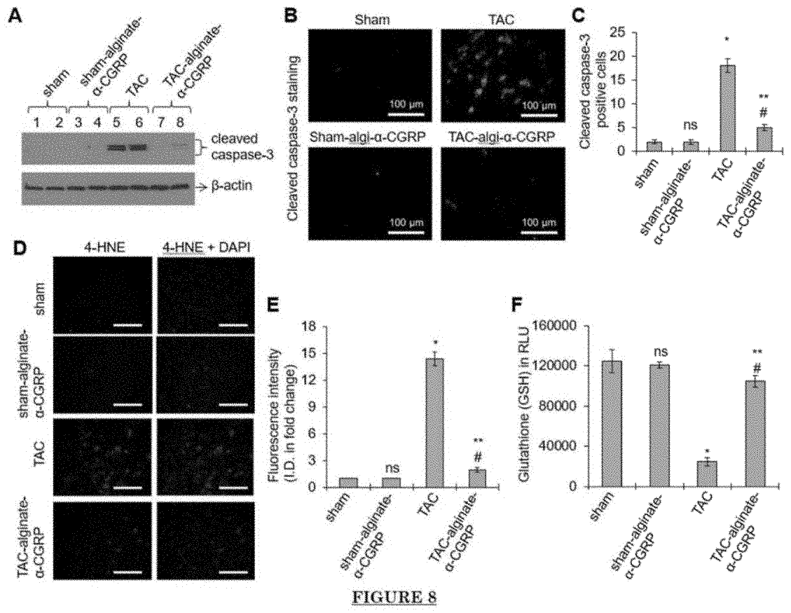

In order to determine if the cardiac cellular damage was also attenuated by alginate-α-CGRP microcapsule treatment, gross and histological measurements were taken of hearts from all of the groups. At the conclusion of the experiment, all groups, treated and sham, were sacrificed. Hearts and lungs were isolated, photographed, and the ratio of wet heart weight, to tibia length and wet lung weight to tibia length were measured as indices of LV hypertrophy and dilation and pulmonary congestion, see FIG. 7 at A-C. The representative photographs and bar diagrams in FIG. 7 at A and B show that hearts from TAC mice were larger than that from the sham mice (*p<0.05, TAC-only vs sham). Additionally, hearts from mice treated with alginate-α-CGRP microcapsules was significantly smaller than TAC (**p<0.05, TAC-alginate-α-CGRP vs TAC) and comparable to sham hearts (#p>0.05, TAC-alginate-α-CGRP vs sham-only; FIG. 7 at A and B). Similarly, the calculated mean lung weight/tibia length was significantly greater in TAC mice compared to sham mice (*p<0.05, TAC vs sham) while the increase in lung weight/tibia length after TAC was significantly reduced by α-CGRP administration (**p<0.05, TAC-alginate-α-CGRP vs TAC-only, see FIG. 7 at C). The lung weight between TAC-alginate-α-CGRP and sham group of mice was not significantly different (#p>0.05. TAC-alginate-α-CGRP vs sham). The heart size and the ratios heart weight/tibia length and lung weight/tibia length among the sham-alginate-α-CGRP mice and sham-only mice appeared nearly identical (ns, sham-alginate-α-CGRP vs sham-only; FIG. 7 at A-C).

To determine the effect of alginate-α-CGRP microcapsule treatment on cardiac myocyte size, H&E staining and wheat germ agglutinin (WGA) staining was performed, see FIG. 7 at D. As expected, the TAC procedure markedly increased myocytes size in the LVs (*p<0.05, TAC vs sham, see FIG. 7 at E). However, LV myocytes size in the TAC-alginate-α-CGRP group was significantly decreased compared to TAC-only mice and was almost identical to sham-only mice (**p<0.05, TAC-alginate-α-CGRP vs TAC-only; and #p>0.05, TAC-alginate-α-CGRP vs sham). Treatment with alginate-α-CGRP microcapsules did not affect LV cardiomyocyte size in sham-alginate-α-CGRP mice when compared to sham LV (ns=nonsignificant vs sham). Likewise, when compared to sham, TAC surgery significantly increased LV fibrosis which was decreased with α-CGRP administration in TAC mice (*p<0.05, TAC vs sham; **p<0.05, TAC-alginate-α-CGRP vs TAC; #p<0.05, TAC-alginate-α-CGRP vs sham, see FIG. 7 at D and F).

α-CGRP Administration Reduces Apoptosis and Oxidative Stress in TAC LVs

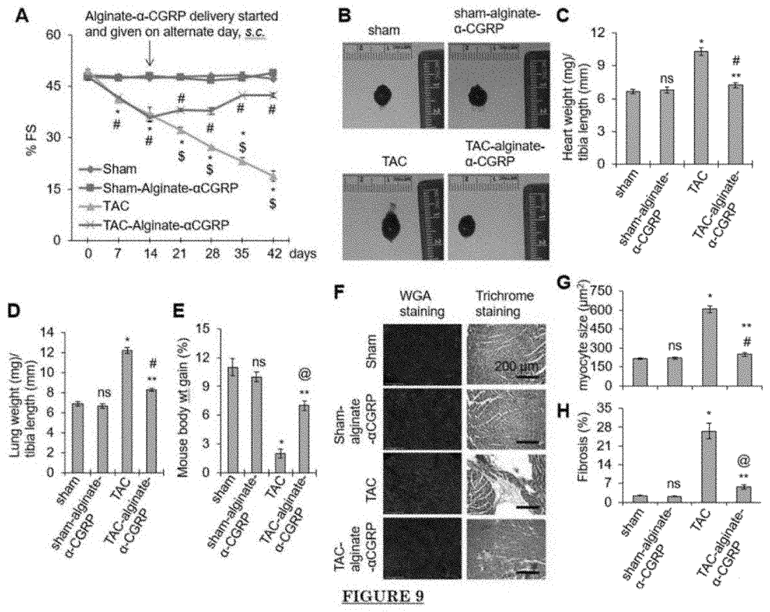

Our previous studies showed that following TAC, there is an increases in cell death and an elevation in oxidative stress markers. We therefore set out to determine if α-CGRP administration could mitigate these responses. Western blot analysis for the presence of apoptosis markers demonstrated that cleaved caspsase-3 (a marker of apoptotic cell death) was significantly higher in TAC LVs compared to sham LV, and alginate-α-CGRP microcapsules administration significantly reduced cleaved caspsase-3 levels to those observed in sham LVs, see FIG. 8 at A. Similarly, the number of cleaved caspase-3 positive cells (green) were higher in TAC LVs when compared to the sham LV (*p<0.05, TAC vs sham, FIG. 8 at B and C). Similarly, when we analyzed the number of cleaved caspase-3 positive cells we determined that it was significantly lower in the TAC-alginate-α-CGRP LVs to TAC LVs and comparable to that of sham IVs (**p<0.05, TAC-alginate-α-CGRP vs TAC; #p<0.05, TAC-alginate-α-CGRP vs sham; FIG. 8 at B and C).

We also examined the hearts for 4-HNE, a marker of oxidative stress-induced lipid-peroxidation. Sections of LVs were images and its immunofluorescence quantitated. We observed that TAC induced pressure-overload markedly increased formation of HNE-adduct in TAC-LV (*p<0.05, TAC vs sham; FIG. 8 at D-E), and α-CGRP administration significantly reduced the intensity of signal of 4-HNE in the TAC LV and was comparable to their sham counterpart (**p<0.05, TAC-alginate-α-CGRP vs TAC; #p<0.05, TAC-alginate-α-CGRP vs sham). FIG. 8 at F showed that the total glutathione level was significantly reduced in the TAC LVs (*p<0.05, TAC vs sham) while significantly restored by treatment of alginate-α-CGRP microcapsules (**p<0.05, TAC-alginate-α-CGRP vs TAC; #p<0.05, TAC-alginate-α-CGRP vs sham). All of the oxidative stress parameters in sham-alginate-α-CGRP LVs were comparable with sham LVs (ns=non-significant compared to sham; FIG. 8 at D-F). These results suggest that α-CGRP delivery through alginate microcapsules protected cardiac cells from pressure-overload induced apoptosis and oxidative stress.

Alginate-α-CGRP Microcapsules Administration Improves Cardiac Function in 15-Day Post TAC-Mice

Our results from these experiments demonstrated that α-CGRP microcapsule delivery, beginning two-day post-TAC, protected mice against adverse pressure-induced cardiac effects. We next wanted to determine if our alginate-α-CGRP microcapsules could ameliorate these effects after the progression of heart failure had already begun. This would move our studies from a preventive approach to an actual treatment approach. To address this, we again performed TAC surgery in mice, and then 15 days after TAC, alginate-α-CGRP microcapsules (containing 150 μg α-CGRP; final α-CGRP dose 6 mg/kg/mouse) were administered s.c. on alternate days for an additional 28 days. Day 15 was chosen as it's a timepoint when all deleterious measures of heart failure are present in mice following TAC surgery. Echocardiogram data showed the usual result that TAC significantly reduced cardiac fraction shortening (FS) (*p<0.05, TAC vs sham). What was exciting was that alginate-α-CGRP microcapsules administration attenuated the reduction in FS following 28 days of treatment. The FS in TAC-alginate-α-CGRP mice was significantly improved compared to TAC mice and was comparable with that of sham mice ($p<0.05, TAC vs TAC-alginate-α-CGRP at the same time point), see FIG. 9 at A. When compared to TAC mice, the wet heart wt and lung wt in TAC-alginate-α-CGRP mice was significantly lower indicating that α-CGRP delivery significantly inhibited cardiac hypertrophy and pulmonary edema in TAC-mice, see FIG. 9 at B-D. During the length of experiment, the TAC group of mice gained only 2% body wt. while sham, sham-alginate-α-CGRP, and TAC-alginate-α-CGRP group of mice gained (in %) 11, 10, and 7 body wt, respectively, indicating that α-CGRP improved body gain in TAC mice, see FIG. 9 at E. Moreover, administration of alginate-α-CGRP microcapsules starting at day 15, significantly attenuated the increased size of cardiomyocytes, see FIG. 9 at F and G, and fibrosis (as determined by collagen content after Masson's trichrome collagen staining; FIG. 9 at F and H) in TAC-IVs after 28 days of treatment. Although O-CGRP concentration used in present study significantly inhibited fibrosis in TAC-LVs, it did not reduce the level to that observed in sham-LVs, see FIG. 9 at H. Our CGRP-treatment study demonstrated, for the first time, that α-CGRP alginate microcapsules administration beginning 15-days post-TAC protected hearts both at physiological and pathological levels and reversed the deleterious effects of pressure overload in heart.

Using genetic and pharmacological approaches, a series of independent studies from our laboratory and other research groups established that α-CGRP deletion makes the heart more vulnerable to heart failure, hypertension, myocardial infarction, and cardiac and cerebral ischemia indicating α-CGRP is protective against various cardiac diseases. Hearts from the α-CGRP KO mice exhibited a significant reduction in cardiac performance following I/R injury due to elevated oxidative stress and cell death when compared with their WT counterparts. A similar cardioprotective role of α-CGRP has been determined in murine models of hypertension including deoxycorticosterone (DOC)-salt, subtotal nephrectomy-salt, L-NAME-induced hypertension during pregnancy, a two-kidney one-clip model of hypertension, and in chronic hypoxic pulmonary hypertension. Moreover, several human and animal studies showed that exogenous delivery of α-CGRP peptide benefits against cardiac diseases. In patients with stable angina pectoris, intracoronary infusion of α-CGRP delayed the onset of myocardial ischemia. Also, in patients with congestive heart failure, an acute intravenous infusion of α-CGRP improves myocardial contractility and thus improving cardiac functions. Similarly, infusion of α-CGRP in patients with heart failure decreased systemic arterial pressure. Our previous study confirmed that long-term administration of native α-CGRP, through osmotic mini-pumps, significantly preserve the hearts at functional and anatomical levels in TAC pressure-overload mice. A similar study using α-CGRP KO mice presented data that supports our findings on the cardioprotective role of α-CGRP in cardiac diseases and showed that native α-CGRP delivery through osmotic mini-pumps corrected adverse effects of hypertension in these KO mice. Furthermore, subcutaneous administration of an acylated version of α-CGRP, a stable α-CGRP agonist, significantly reduced cardiac hypertrophy, fibrosis, inflammation and oxidative stress in rodent models of hypertension and heart failure. Together, these studies establish α-CGRP as a promising drug candidate to treat and prevent cardiovascular diseases. However, the low bioavailability of the native peptide in human plasma (t_{1/2}=˜5.5 min) makes it difficult to use α-CGRP as a therapeutic agent in a long term treatment regime. Moreover, the applicability of osmotic mini-pump as a peptide delivery system is not feasible in humans. In light of this, new approaches are warranted if α-CGRP is to be an effective and accessible treatment for heart failure.

The present study demonstrated that using an alginate polymer as a drug carrier for α-CGRP was effective in ameliorating pressure-overload induced heart failure. Moreover, cell apoptosis and oxidative stress that accompanies worsening heart failure was reduced by the treatment with alginate-α-CGRP microcapsules. Several lines of evidence demonstrated that systemic administration of α-CGRP reduces BP, however, the reduction in blood pressure is very short because the half-life of native α-CGRP in human plasma is only 5.5 min. We previously used alginate microencapsulation to treat numerous ocular and skin wounds. Recently we used cellular alginate microencapsulation to treat and improve the symptoms of macular degeneration in a mouse model. Alginate is a natural polysaccharide extracted from seaweeds and has been extensively used to encapsulate a wide range of molecules-ranging from large macromolecules, such as cells, DNA and protein, to small molecules—peptides and antibodies. In the current study we developed a novel alginate based α-CGRP delivery system to deliver α-CGRP in controlled and sustained manner. Our state-of-art technology used an electrospray method to prepare α-CGRP encapsulated alginate microcapsules of a consistent size and release. The advantage of using an electrospray method is that the alginate-α-CGRP capsules can range from nano- to micro-size (ranging from 10 nm-500 μm) by adjusting the experimental parameters, e.g., the voltage, flow rate, and distance between needle to gelling bath solution. In addition, one can modify the microcapsule to release its contents at the desired interval.

Encapsulated microcapsules are very stable at room temperature as the spherical shape of alginate-alone and alginate-α-CGRP microcapsules in deionized water was remained intact even after 15 months (data not shown). Encapsulated peptide remained biologically active in vivo as released α-CGRP from subcutaneously administered alginate-α-CGRP microcapsules lowered the BP, an inherent property of native α-CGRP, in mice, see FIG. 4. Also, alginate-α-CGRP microcapsule formulation is non-toxic to cardiac cells, see FIG. 5 at E and F. Alginate-α-CGRP microcapsules upto 5 μM (maximum concentration tested) did not affect the growth of H9C2 cells, see FIG. 5 at E. Similarly, HL-1 cells kept beating on the plate even after 1 h incubation with 10 μM alginate-α-CGRP microcapsules, see FIG. 5 at F. These data indicated that alginate-α-CGRP microcapsules neither affect viability nor beating phenotype of cardiac cells under in vitro conditions.

Another important finding of the study is that alginate-α-CGRP microcapsules (containing 150 μg α-CGRP; final α-CGRP dose 6 mg/kg/mouse) subcutaneously administered in pressure-overload heart failure mice, improved myocardial function by restoring both FS and EF, hallmarks of increasing heart failure and attenuated increased apoptotic cell death and oxidative stress in TAC-LVs.

Previously, it has been shown that intravenous injections of α-CGRP significantly decreases mean arterial pressure (MAP) in a dose-dependent fashion in both normal and spontaneously hypertensive rats, however. MAP returns to normal baseline after 20 min of injection in both groups of rats. Our findings demonstrated that subcutaneous administration of 150 μg and 250 μg of alginate-α-CGRP microcapsules per 25 g mouse lowered the systolic pressure for 18 h and 3 days, respectively. Moreover, our results indicate that addition of alginate-α-CGRP microcapsules extends the release of peptide, and released α-CGRP remains biologically active for extended periods of time.

Another novel and exciting finding of the present study is that when alginate microcapsules were administered starting at 15-day post-TAC mice there was an immediate reversal of symptoms. This was similar to the ability of α-CGRP filled alginate microcapsules to significantly protect hearts when administered immediately after surgery. Also similar to early administration, treatment started at 15 days post TAC was able to reverse all of the parameters of heart failure examined to include, cardiac hypertrophy, apoptosis, cardiac function and fibrosis. This is the first demonstration that addition of α-CGRP just prior to the onset of symptoms could reverse quickly the damage that is observed with TAC induced heart failure.

Alginate is non-toxic and immunologically inactive, hence prepared alginate based drug formulation does not exhibit side effects and has been FDA approved for use in humans. Our laboratory has established that alginate microcapsules can also undergo freeze-thaw cycles as well as can be lyophilized without compromising the integrity of microcapsules (Data not shown). The lyophilized form of alginate microcapsules immediately swell and regain their shape when suspended in distilled water. Consequently, alginate-α-CGRP microcapsules can be stored at very low temperature and lyophilized to make their easy transport. With these advantages, alginate-α-CGRP microcapsules can be employed as an effective way for controlled and sustained delivery of α-CGRP in humans suffering from cardiovascular diseases. The success of this novel drug delivery technology will have the potential to dramatically change conventional drug therapies used presently to treat the failing heart.

All together these data indicate that an alginate microcapsules based delivery system is an effective strategy to improve α-CGRP bioavailability in plasma and, thus, increase the duration of the therapeutic effect of the peptide throughout the treatment period. In addition, the observed cardioprotective effects of alginate-α-CGRP microcapsules was present either administering prior to symptoms (ie. CGRP-prevention study) or at 15 days post-TAC when symptoms are beginning (ie. CGRP-treatment study). Thus our study suggests that the developed alginate-α-CGRP microcapsule administration can be effective in the prevention and represents a new treatment of heart failure.

### FIGURE LEGENDS

FIG. 6 at A—Representative echocardiograms showing short axis B- and M-mode 2D echocardiography performed after 28 days delivery of alginate-α-CGRP microcapsules in sham and TAC-mice. Percentage fractional shortening (FS) and ejection fraction (EF) was calculated at various time points and plotted (B and C).

FIG. 7 at A—Representative images showing the size of the hearts after 28 days delivery of alginate-α-CGRP microcapsules. (B and C)—Bar diagrams showing the ratio of wet heart weight/tibia length, and wet lung weight/tibia length. (D)—The paraffin-embedded LV sections were stained with H&E, WGA stain, and Trichrome-collagen stain. Scale bar=100 μm. WGA stained sections were used to measure cardiomyocyte size in LVs by NIH-ImageJ software and plotted (E). LV collagen content, an indicator of fibrosis, was quantitated by NIH-ImageJ software and plotted (F). Values were expressed as the mean±SEM. *p<0.05, TAC vs sham; **p<0.05, TAC-alginate-α-CGRP vs TAC; #p>0.05, TAC-alginate-α-CGRP vs sham; ns=non-significant compared to sham.

FIG. 8 at A—Western blot showing level of cleaved caspase-3 protein in LVs from sham, sham-alginate-α-CGRP, TAC, and TAC-alginate-α-CGRP. β-actin was used as control. (B)— Representative fluorescence images showing cleaved caspase-3 staining (green) to detect apoptosis in the LV sections. Scale=100 μm. Cleaved caspase-3 positive cells (green) were counted and plotted as the mean SEM (C). (D and E)-Fluorescence images showing 4-HNE staining (a marker of lipid peroxidation) in the paraffin-embedded LV sections. DAPI was used to stain nuclei. Scale=100 lim. The fluorescence intensity of 4-HNE (red) was quantitated by NIH-ImageJ software and plotted as the mean±SEM. I.D.=integrated density. (F)— Bar diagrams showing glutathione (GSH) level in the IVs. Values were expressed as the mean±SEM and p<0.05 was considered significant. *p<0.05, TAC vs sham; **p<0.05, TAC-alginate-α-CGRP vs TAC; #p>0.05. TAC-alginate-α-CGRP vs sham; ns=not-significant compared to sham.

FIG. 9 at A—Graph showing % FS in sham, sham-alginate-α-CGRP, TAC-only, and TAC-alginate-α-CGRP groups of mice. After 15 days of TAC, alginate-α-CGRP microcapsules (α-CGRP dose 6 mg/kg/mouse) were injected on alternate day, till day 28. Echocardiography was performed at different time points and % FS was plotted as mean±SEM. *p<0.05. TAC vs sham at the same time point; #p<0.05. TAC-alginate-α-CGRP vs sham at the same time point; Sp<0.05, TAC vs TAC-alginate-α-CGRP at the same time point. (B). Representative images showing the size of hearts after 28 days delivery of alginate-α-CGRP microcapsules. Ratio of wet heart weight/tibia length was plotted as mean±SEM (C). (D)—Bar diagram showing ratio of wet lung weight/tibia length as mean±SEM. (E)—Bar diagram showing mice weight gain (in percentage) during the course of experiment as mean±SEM. p<0.05 was considered significant. *p<0.05, TAC vs sham; **p<0.05, TAC-alginate-α-CGRP vs TAC; #p>0.05, TAC-alginate-α-CGRP vs sham; ^{@}p<0.05, TAC-alginate-α-CGRP vs sham; ns=not-significant compared to sham. (F)—Representative histology images showing size of cardiomyocytes (WGA staining) and level of fibrosis (trichrome-collagen staining) in the LVs from different groups of mice. Cardiomyocyte size (G) and % fibrosis (H) in LVs was quantitated using NTH-ImageJ software and plotted as mean±SEM. p value<0.05 was considered significant. *p<0.05. TAC vs sham; **p<0.05, TAC-alginate-α-CGRP vs TAC; #p>0.05, TAC-alginate-α-CGRP vs sham; **p<0.05, TAC-alginate-α-CGRP vs sham; ns=not-significant compared to sham.

Amino Acid Sequences

A)—Peptide Human α-CGRP Amino Acid Sequence—

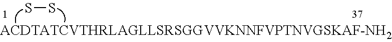

Sequence Listing Free Text

Ala-Cys-Asp-Thr-Ala-Thr-Cys-Val-Thr-His-Arg-Leu-
Ala-Gly-Leu-Leu-Ser-Arg-Ser-Gly-Gly-Val-Val-Lys-
Asn-Asn-Phe-Val-Pro-Thr-Asn-Val-Gly-Ser-Lys-Ala-
Phe-NH2

B)—Peptide Rodent (Mouse or Rat) α-CGRP Amino Acid Sequence—

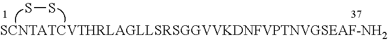

Sequence Listing Free Text

Ser-Cys-Asn-Thr-Ala-Thr-Cys-Val-Thr-His-Arg-Leu-
Ala-Gly-Leu-Leu-Ser-Arg-Ser-Gly-Gly-Val-Val-Lys-
Asp-Asn-Phe-Val-Pro-Thr-Asn-Val-Gly-Ser-Glu-Ala-
Phe-NH2

Sequence Legend: Human α-CGRP amino acid sequence (A) and rodent (mouse or rat) α-CGRP (B) have an identical amino acid sequence except at four amino acid positions—1, 3, 25, and 35. However both, human and rodent (mouse or rat) α-CGRPs, share identical biological activities. Human α-CGRP (A) and rodent α-CGRP (B) are a single peptide of 37-amino acids containing one disulfide bond (—S—S—) between amino acids 2 and 7 (cys2-cys7) and one amide molecule (—NH2) at the C-terminal end. Positions of the first and last amino acid in each peptide sequence is marked as 1 and 37, respectively.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A novel delivery system for maintaining peptide levels in plasma comprising:
at least one α-CGRP peptide;
at least one alginate polymer;
wherein the at least one α-CGRP peptide is encapsulated in the at least one alginate polymer to form at least one alginate-α-CGRP peptide.

2. The delivery system of claim 1, wherein the delivery system releases the at least one α-CGRP peptide over time to maintain a constant level of the at least one α-CGRP peptide in plasma.

3. The delivery system of claim 1, wherein the at least one α-CGRP peptide remains biologically active after encapsulation.

4. The delivery system of claim 1, wherein the at least one α-CGRP peptide is encapsulated via an electrospray method.

5. The delivery system of claim 1, wherein the at least one alginate-α-CGRP peptide remains stable for up to one year at room temperature.

6. The delivery system of claim 1, wherein the at least one alginate-α-CGRP peptide lowers blood pressure.

7. The delivery system of claim 1, wherein the system is tunable to arrive at a pre-selected dosage of the at least one α-CGRP peptide delivered over an extended period of time.

8. The delivery system of claim 1, wherein the at least one alginate polymer comprises sodium-alginate.

9. The delivery system of claim 1, wherein the at least one alginate-α-CGRP peptide is introduced via subcutaneous administration.

10. The delivery system of claim 1, wherein the at least one α-CGRP peptide is replaced with at least one α-CGRP peptide agonist analog.

11. A method for forming an alginate-based drug delivery system comprising:
suspending at least one alginate polymer in a liquid;
preparing a stock solution of at least one α-CGRP peptide;
preparing an ionic gelling bath solution;
mixing the at least one alginate polymer and the at least one at least one α-CGRP peptide to form a mixture;
flowing the mixture through a charge into the ionic gelling bath solution to encapsulate the at least one α-CGRP peptide in the at least one alginate polymer to form at least one alginate-α-CGRP peptide microcapsule.

12. The method of claim 10, wherein the at least one alginate-αCGRP microcapsule is formed to be introduced via subcutaneous administration.

13. The method of claim 10, wherein the ionic gelling batch solution comprises calcium chloride.

14. The method of claim 10, further comprising coating the at least one alginate-α-CGRP peptide microcapsule with at least one amino acid chain.

15. The method of claim 14, wherein the at least one amino acid chain is poly-L-ornithine or poly-L-lysine.

16. The method of claim 10, further comprising irradiating the at least one alginate-α-CGRP peptide microcapsule with ultraviolet light.

17. The method of claim 10, wherein size of the at least one alginate-α-CGRP peptide microcapsule is be adjusted via modifying voltage, flow rate, and/or distance to the gelling bath solution.

18. The method of claim 10, further comprising coating the at least one alginate-α-CGRP peptide microcapsule with chitosan.


SEQUENCE LISTING (2 sequences)

SEQ ID NO: 1 - protein, 37 aa, Homo sapiens
ACDTATCVTHRLAGLLSRSGGVVKNNFVPTNVGSKAF

SEQ ID NO: 2 - protein, 37 aa, Mus musculus
SCNTATCVTHRLAGLLSRSGGVVKDNFVPTNVGSEAF